(19) United States
(12) Patent Application Publication
(10) Pub. No.: US 2022/0235022 A1
(43) Pub. Date: Jul. 28, 2022

(54) METHODS FOR PREPARING CANNABINOIDS BY BASE-PROMOTED DOUBLE-BOND MIGRATION

(71) Applicant: Canopy Growth Corporation, Smiths Falls (CA)

(72) Inventors: Christopher ADAIR, Smiths Falls (CA); Ben GEILING, Smiths Falls (CA); Mohammadmehdi HAGHDOOST MANJILI, Smiths Falls (CA)

(21) Appl. No.: 17/596,341

(22) PCT Filed: Jun. 11, 2020
(86) PCT No.: PCT/CA2020/050805

§ 371 Date: Dec. 8, 2021

Related U.S. Application Data
(60) Provisional application No. 62/860,172, filed on Jun. 11, 2019.

(51) Int. Cl. C07D 311/80; C07C 37/00

(52) U.S. Cl. CPC C07D 311/80; C07C 37/00

(57) ABSTRACT

Disclosed is a method for converting a first cannabinoid into a second cannabinoid that is a regioisomer of the first cannabinoid. The method comprising contacting the first cannabinoid with: (i) a base having a pKb that is less than a critical pKb for the first cannabinoid; and (ii) a solvent system comprising a polar solvent such as dimethyl sulfoxide (DMSO), triethylamine (TEA), or a combination thereof.

### CROSS-REFERENCE TO RELATED APPLICATION

This application claims priority to and benefit of U.S. Provisional Patent Application Ser. No. 62/860,172 filed on Jun. 11, 2019, which is hereby incorporated by reference.

### TECHNICAL FIELD

The present disclosure generally relates to methods for isomerizing cannabinoids. In particular, the present disclosure relates to methods for preparing cannabinoids by inducing double-bond migration reactions with basic reagents.

### BACKGROUND

Cannabinoids are often defined in pharmacological terms as a class of compounds that exceed threshold-binding activities for specific receptors found in central-nervous-system and/or peripheral tissues. Such pharmacological definitions are functional in nature, and they encompass a wide range of compounds with, for example: various structural forms (e.g. different fused-ring systems); various functional-group locants (e.g. different arene-substitution patterns); and/or various alkyl-substituent chain lengths (e.g. C₃H₇ vs C₅H₁₁). Accordingly, cannabinoids are also often defined based on chemical structure and, in this context, many cannabinoids are characterized as isomeric cannabinoids. Isomeric cannabinoids are those which share the same atomic composition but different structural or spatial atomic arrangements. For example, Δ¹-cannabidiol (Δ¹-CBD), Δ⁶-cannabidiol (Δ⁶-CBD), Δ⁸-tetrahydrocannabinol (Δ⁸-THC), Δ⁹-tetrahydrocannabinol (Δ⁹-THC), and Δ¹⁰-tetrahydrocannabinol (Δ¹⁰-THC) are all isomeric cannabinoids in that they each have an atomic composition of C₂₁H₃₀O₂, but different structural arrangements as shown in SCHEME 1:

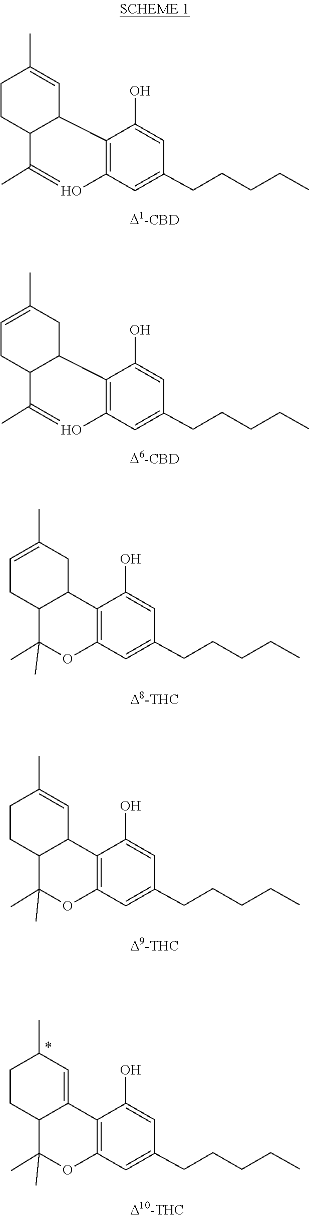 [SMILES: C=C(C)C1CCC(C)=CC1c1c(O)cc(CCCCC)cc1O ; C=C(C)C1CC=C(C)CC1c1c(O)cc(CCCCC)cc1O ; CCCCCc1cc(O)c2c(c1)OC(C)(C)C1CC=C(C)CC21 ; CCCCCc1cc(O)c2c(c1)OC(C)(C)C1CCC(C)=CC21 ; CCCCCc1cc(O)c2c(c1)OC(C)(C)C1CCC(C)C=C21]

Compounds that differ only in the location of a particular functional group are known as regioisomers. Hence, cannabinoids that differ only in the location of a particular functional group are known as regioisomeric cannabinoids. Δ⁶-CBD and Δ¹-CBD are archetypal regioisomer cannabinoids as are Δ⁸-THC, Δ⁹-THC, Δ¹⁰-THC—in both cases their structures differ only in the location of an alkene functional group. Notably, the cannabinoid-receptor-binding affinity for Δ⁸-THC is similar to that of Δ⁹-THC, but Δ⁸-THC is reported to be approximately 50% less potent in terms of psychoactivity. More generally, regioisomeric cannabinoids often have substantially different pharmacological properties, which makes methods for preparing and isolating them desirable—especially because regioisomeric cannabinoids often vary greatly with respect to natural abundance.

### SUMMARY

The present disclosure provides methods for preparing cannabinoids by double-bond-migration reactions wherein a first cannabinoid is converted into a second cannabinoid that is a regioisomer of the first cannabinoid. Importantly, the methods of the present disclosure may provide access to one or more cannabinoids that are not naturally abundant in typical cannabis cultivars. Also importantly, the present disclosure provides methods for preparing mixtures of cannabinoid regioisomers in various relative proportions.

In providing access to: (i) one or more cannabinoids that are not naturally abundant in typical cannabis cultivars; and/or (ii) mixtures of cannabinoid regioisomers in various relative proportions, the methods of the present disclosure employ reaction conditions that are safer, less expensive, and/or operationally more simplistic than those known in the art. The present disclosure posits that these features are engendered by the combination of: (i) a sufficiently basic reagent; and (ii) a solvent system that is sufficiently polar to facilitate acid/base and/or electron-transfer reactions. Dimethylsulfoxide (DMSO) and triethylamine (TEA) are archetypal solvents that facilitate acid/base and/or electron-transfer reactions. As components of a solvent system for acid/base and/or electron-transfer reactions, DMSO and TEA have the potential to modulate reactivity in peculiar ways (i.e. DMSO and TEA often correlate with unusual solvent effects). The methods of the present disclosure take advantage of such unusual solvent effects to facilitate base-promoted double-bond isomerization reactions that convert select cannabinoids into their regioisomeric analogs (or to mixtures of cannabinoids comprising regioisomeric analogs).

In select embodiments, the present invention relates to a method for converting a first cannabinoid into a second cannabinoid that is a regioisomer of the first cannabinoid. In such embodiments, the method comprises contacting the first cannabinoid with a solvent system comprising a polar solvent (such as DMSO and/or TEA) and a base having a pKb of less than a critical pKb for the first cannabinoid.

Other aspects and features of the present disclosure will become apparent to those ordinarily skilled in the art upon review of the following description of specific embodiments.

### BRIEF DESCRIPTION OF THE DRAWINGS

These and other features of the present disclosure will become more apparent in the following description in which reference is made to the appended drawings. The appended drawings illustrate one or more embodiments of the present disclosure by way of example only and are not to be construed as limiting the scope of the present disclosure.

FIG. 1A shows an HPLC-DAD chromatogram of an output material for an attempted double-bond migration reaction in the absence of a suitable solvent system.

FIG. 1B shows an HPLC-DAD chromatogram of an output material for a double-bond migration reaction in accordance with the present disclosure.

FIG. 1C shows an HPLC-DAD chromatogram of an output material for a double-bond migration reaction in accordance with the present disclosure.

FIG. 1D shows an HPLC-DAD chromatogram of an output material for a double-bond migration reaction in accordance with the present disclosure.

FIG. 2A shows an HPLC-DAD chromatogram of an output material for a double-bond migration reaction in accordance with the present disclosure.

FIG. 2B shows an HPLC-DAD chromatogram of an output material for a double-bond migration reaction in accordance with the present disclosure.

FIG. 2C shows an HPLC-DAD chromatogram of an output material for a double-bond migration reaction in accordance with the present disclosure.

FIG. 2D shows an HPLC-DAD chromatogram of an output material for a double-bond migration reaction in accordance with the present disclosure.

FIG. 3A shows an ORTEP drawing of cis-Δ¹⁰-THC produced via a double-bond migration reaction in accordance with the present disclosure.

FIG. 3B shows an ORTEP drawing of trans-Δ¹⁰-THC produced via a double-bond migration reaction in accordance with the present disclosure.

FIG. 3C shows a ¹H NMR spectrum of cis-Δ¹⁰-THC produced via a double-bond migration reaction in accordance with the present disclosure.

FIG. 3D shows a ¹H NMR spectrum of trans-Δ¹⁰-THC produced via a double-bond migration reaction in accordance with the present disclosure.

FIG. 3E shows a ¹³C NMR spectrum of trans-Δ¹⁰-THC produced via a double-bond migration reaction in accordance with the present disclosure.

FIG. 3F shows a heteronuclear single quantum coherence spectroscopy (HSQC) NMR spectrum of trans-Δ¹⁰-THC produced via a double-bond migration reaction in accordance with the present disclosure.

FIG. 3G shows a heteronuclear multiple bond correlation (HMBC) NMR spectrum of trans-Δ¹⁰-THC produced via a double-bond migration reaction in accordance with the present disclosure.

FIG. 3H shows a mass spectrum of cis-Δ¹⁰-THC produced via a double-bond migration reaction in accordance with the present disclosure.

FIG. 3I shows a mass spectrum of trans-Δ¹⁰-THC produced via a double-bond migration reaction in accordance with the present disclosure.

### DETAILED DESCRIPTION

The present disclosure provides methods for preparing cannabinoids by double-bond-migration reactions wherein a first cannabinoid is converted into a second cannabinoid that is a regioisomer of the first cannabinoid. Importantly, the methods of the present disclosure may provide access to one or more cannabinoids that are not naturally abundant in typical cannabis cultivars. For example, methods of the present disclosure provide access to Δ⁶-cannabidiol (Δ⁶-CBD) and Δ¹⁰-tetrahydrocannabinol (Δ¹⁰-THC) which are less naturally abundant than Δ¹-cannabidiol (Δ¹-CBD) and Δ⁹-tetrahydrocannabinol (Δ⁹-THC), respectively, in many cannabis cultivars. Also importantly, the present disclosure provides methods for preparing mixtures of cannabinoid regioisomers in various relative proportions. While pharma-kinetic interactions between mixtures of cannabinoid regioisomers are not well understood, it is expected that access to an array of compositions of wide ranging regioisomeric ratios is useful in both medicinal and recreational contexts. Moreover, it is expected that access to an array of compositions of varying regioisomeric ratios is useful to synthetic chemists.

In providing access to: (i) one or more cannabinoids that are not naturally abundant in typical cannabis cultivars; and/or (ii) mixtures of cannabinoid regioisomers in various relative proportions, the methods of the present disclosure employ reaction conditions that are safer, less expensive, and/or operationally more simplistic than those known in the art. As noted above, the present disclosure posits that these features are engendered by the combination of: (i) a sufficiently basic reagent; and (ii) a solvent system that comprises a sufficiently polar solvent. Without being bound to any particular theory, the present disclosure asserts that the presence of polar solvent increases the rate of the reaction via unusual solvent effects. Non-exclusive examples of unusual solvent effects include breaking up aggregates of the basic reagent, facilitating electron transfer, and/or selectively solvating a cation associated with the basic reagent to increase the extent of dissociation of the cation from the basic reagent. Polar solvents that include hydrogen-bond accepting functional groups may be particularly effective at increasing the rate of reaction. Non-exclusive examples of polar solvents containing hydrogen-bond accepting groups include dimethylsulfoxide (DMSO) and trimethylamine (TEA). As a component of a solvent system for acid/base and/or electron-transfer reactions, DMSO and/or TEA have the potential to modulate reactivity in peculiar ways (i.e. DMSO and TEA often correlate with unusual solvent effects). For example, reports in the academic literature suggest that (under particular conditions) the strong base, potassium tert-butoxide, reacts with DMSO to form the dimsyl anion which is known to act an electron donor to appropriate substrates (see, e.g., J. Am. Chem. Soc. 2016, 138, 7402-7410).

The methods of the present disclosure take advantage of such unusual solvent effects to facilitate base-promoted double-bond isomerization reactions that convert select cannabinoids into their regioisomeric analogs (or to mixtures of cannabinoids comprising regioisomeric analogs).

In select embodiments, the present disclosure relates to a method for converting a first cannabinoid into a second cannabinoid. The second cannabinoid is a regioisomer of the first cannabinoid. As such, the first cannabinoid and the second cannabinoid are compounds of the same cannabinoid subclass. The method comprises contacting the first cannabinoid with a solvent system comprising a polar solvent and a base having a pKb of less than a critical pKb for the first cannabinoid.

In the context of the present disclosure, the term “contacting” and its derivatives is intended to refer to bringing the first cannabinoid and the solvent system comprising the polar solvent and the base into proximity such that a chemical reaction can occur. In some embodiments of the present disclosure, the contacting may be by adding the first cannabinoid to the solvent system. In some embodiments, the contacting may be by adding the solvent system to the first cannabinoid. In some embodiments, the contacting may be by combining, mixing, or both.

In select embodiments, the polar solvent comprises at least one hydrogen-bond accepting group. Hydrogen bond accepting groups may increase the rate of reaction relative to polar solvents lacking hydrogen bond accepting groups. In select embodiments, the polar solvent is DMSO, TEA, or a combination thereof. DMSO and TEA are class III solvents. A skilled person, having the benefit of the present disclosure, will appreciate that other class III solvents include hydrogen-bond accepting groups.

As used herein, the term “cannabinoid” refers to: (i) a chemical compound belonging to a class of secondary compounds commonly found in plants of genus cannabis, (ii) synthetic cannabinoids and any enantiomers thereof; and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In select embodiments of the present disclosure, the cannabinoid is made in a laboratory setting, sometimes called a synthetic cannabinoid. In one embodiment, the cannabinoid is derived or obtained from a natural source (e.g. plant) but is subsequently modified or derivatized in one or more different ways in a laboratory setting, sometimes called a semi-synthetic cannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form). As will be appreciated by those skilled in the art who have benefitted from the teachings of the present disclosure, the terms “first cannabinoid” and/or “second cannabinoid” may refer to: (ii) salts of acid forms, such as Na⁺ or Ca²⁺ salts of such acid forms; and/or (iii) ester forms, such as formed by hydroxyl-group esterification to form traditional esters, sulphonate esters, and/or phosphate esters.

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC), Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11 nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-C5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

Within the context of this disclosure, where reference is made to a particular cannabinoid without specifying if it is acidic or neutral, each of the acid and/or decarboxylated forms are contemplated as both single molecules and mixtures.

As used herein, the term “THC” refers to tetrahydrocannabinol. “THC” is used interchangeably herein with “Δ9-THC”.

In select embodiments of the present disclosure, a “first cannabinoid” and/or a “second cannabinoid” may comprise THC (Δ9-THC), Δ8-THC, trans-Δ10-THC, cis-Δ10-THC, THCV, Δ8-THCV, Δ9-THCV, CBD, CBDA, CBDV, CBDVA, CBC, CBCA, CBCV, CBG, CBGV, CBN, CBNV, CBND, CBNDV, CBE, CBEV, CBL, CBLV, CBT, or cannabicitran.

Structural formulae of cannabinoids of the present disclosure may include the following:

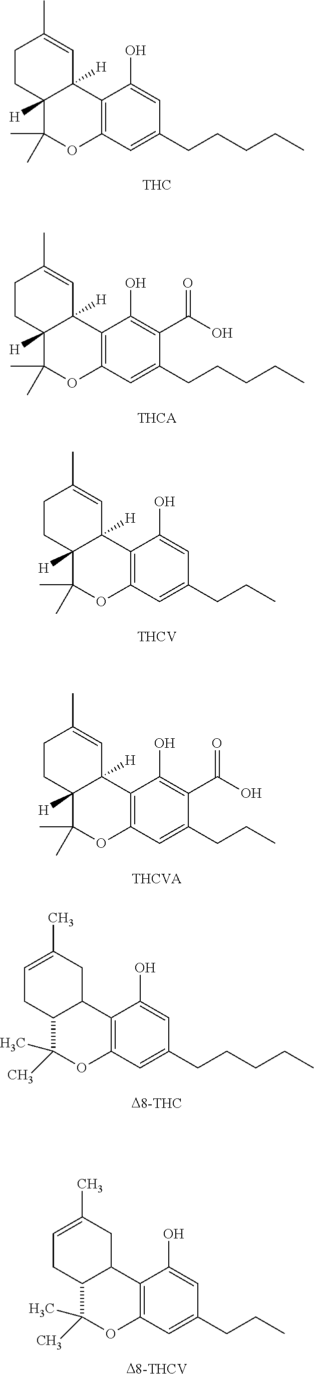

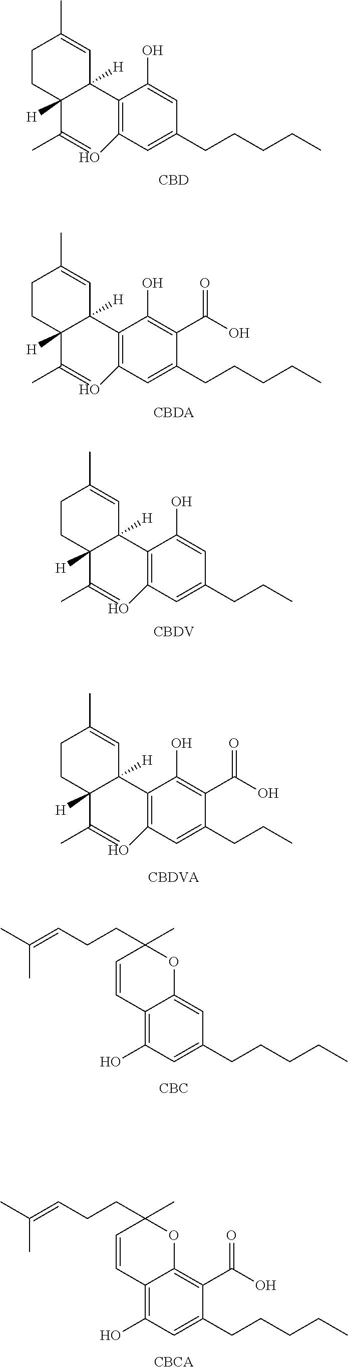

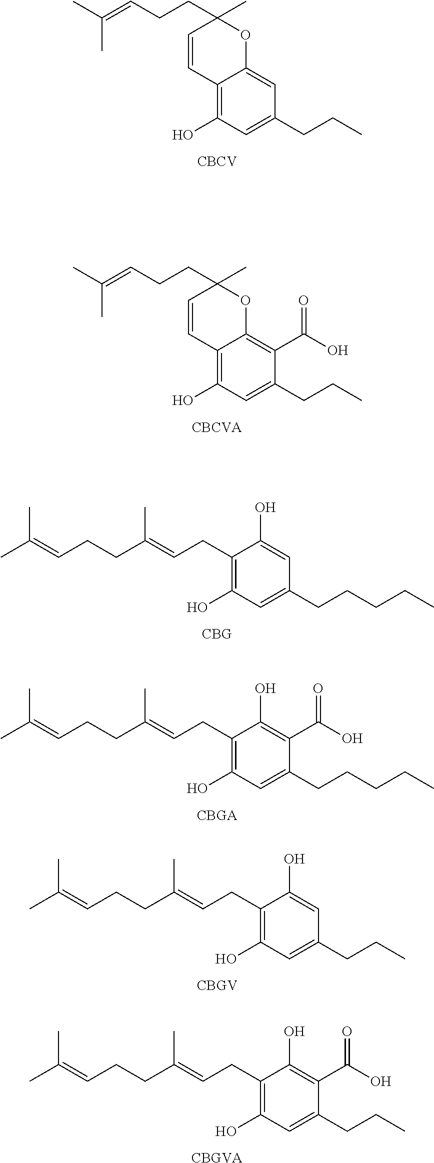

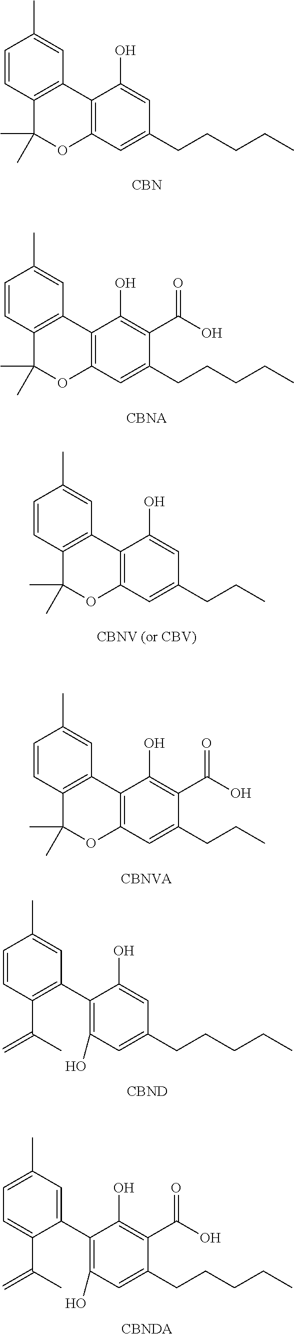

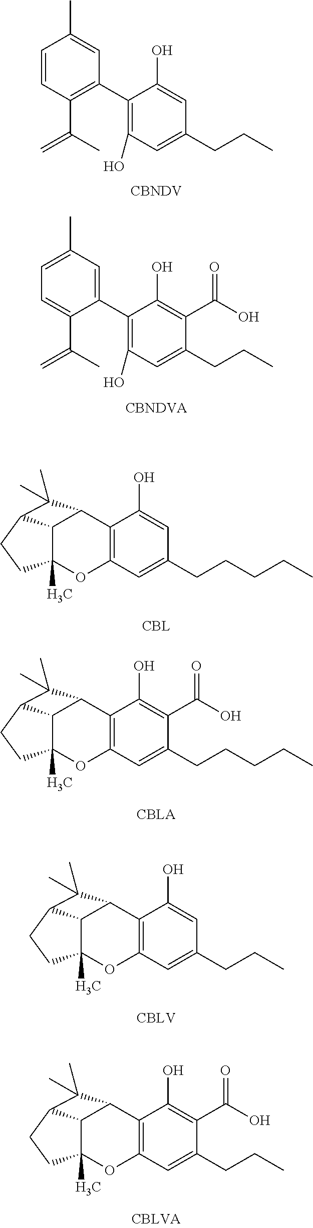

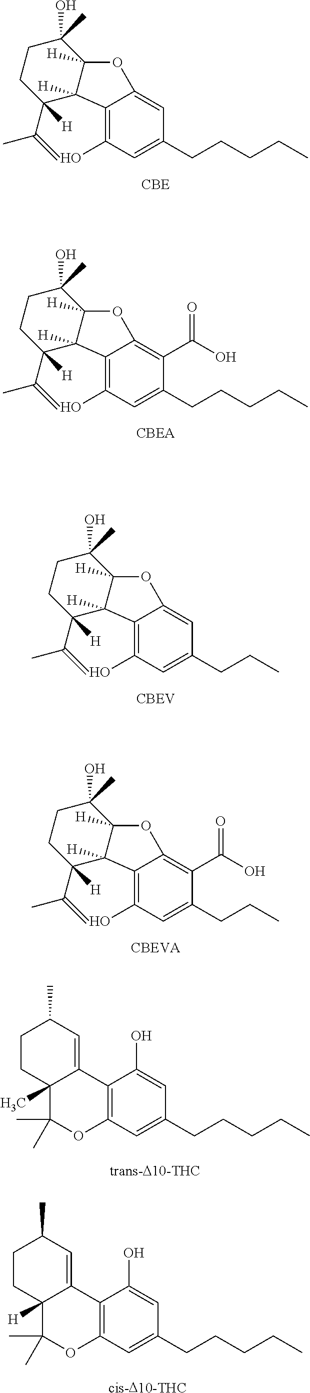

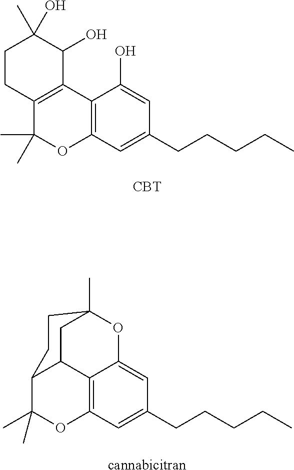

In select embodiments of the present disclosure, the “first cannabinoid” or the “second cannabinoid” may comprise CBD, CBDV, CBC, CBCV, CBG, CBGV, THC, THCV, or a regioisomer thereof. As used herein, the term “regioisomers” refers to compounds that differ only in the location of a particular functional group.

In select embodiments of the present disclosure, the first cannabinoid is Δ⁹-THC or Δ¹⁰-THC.

In select embodiments, the first cannabinoid is a component of a distillate, an isolate, a concentrate, an extract, or a combination thereof.

In the context of the present disclosure, the relative quantities of a first cannabinoid and a second cannabinoid in a particular composition may be expressed as a ratio—second cannabinoid:first cannabinoid. In select embodiments of the present disclosure, a first cannabinoid may be converted into a mixture of cannabinoid products referred to herein as a second cannabinoid, a third cannabinoid, and so on. The relative quantities of cannabinoid products in a mixture may be referred to with analogous ratios (e.g. second cannabinoid:third cannabinoid). Those skilled in the art will recognize that a variety of analytical methods may be used to determine such ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. In select embodiments of the present disclosure, the compositions provided by the methods of the present disclosure have second cannabinoid:first cannabinoid ratios of greater than 1.0:1.0, meaning the quantity of the second cannabinoid in the composition is greater than the quantity of the first cannabinoid in the composition. For example, the compositions provided by the methods of the present disclosure may have second cannabinoid:first cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0. In select embodiments of the present disclosure, the compositions provided by the methods of the present disclosure have second cannabinoid:third cannabinoid ratios of greater than 1.0:1.0 meaning the quantity of the second cannabinoid in the composition is greater than the quantity of the third cannabinoid in the composition. For example, the compositions provided by the methods of the present disclosure may have second cannabinoid:third cannabinoid ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; and (viii) greater than about 100.0:1.0.

As used herein, the term “base” refers to a material that has a pKb that is less than a critical pKb for the first cannabinoid. As used herein, the “critical pKb” for particular cannabinoid is the point at which the base is sufficiently basic to promote a double-bond-isomerization reaction having regard to the effects of the solvent system. pKb data for a number of bases in accordance with the present disclosure are set out in TABLE 1. In considering the data in TABLE 1, those skilled in the art who have benefitted from the teachings of the present disclosure will appreciate that base strength is typically reported as the pKa of the conjugate acid in the literature and that pKb values may be calculated by EQN 1.

pKb=13.9965−pKa EQN. 1

Those skilled in the art who have benefitted from the teachings of the present disclosure will also appreciate that counter ions influence basicity such that lithium-, sodium-, and potassium-coordinated bases may have different pKb values. Accordingly, the values in TABLE 1 should be considered as approximates intended to facilitate the skilled person practicing the methods of the present disclosure.

TABLE 1
pKb-related data for a series of bases in accordance with the present disclosure.

| | pKa | | pKa | |
|---|---|---|---|---|
| | (conjugate | | (conjugate | |
| | acid in | | acid in | Potential Solvent |
| Base | water) | pKb | DMSO) | Combinations |
| tert- | 53 | −39 | n.d. | Diethyl ether, diglyme, |
| butyllithium | | | | DMSO, dioxane, heptane, |
| | | | | pentane, TBME, THF |
| sec- | 51 | −37 | n.d. | Diethyl ether, diglyme, |
| butyllithium | | | | DMSO, dioxane, heptane, |
| | | | | pentane, TBME, THF |
| n-butyllithium | 50 | −36 | n.d. | Diethyl ether, diglyme, |
| | | | | DMSO, dioxane, heptane, |
| | | | | pentane, TBME, THF |
| Methyl | 48 | −34 | n.d. | Diethyl ether, diglyme, |
| Magnesium | | | | DMSO, dioxane, heptane, |
| Bromide/ | | | | pentane, TBME, THF |
| Chloride | | | | |
| Sodium/ | 36 | −22 | n.d. | Diethyl ether, diglyme, |
| Potassium | | | | DMSO, dioxane, heptane, |
| Hydride | | | | pentane, TBME, THF, toluene |
| Lithium | 35.7 (THF) | −21.7 | 22.5 | Diethyl ether, diglyme, |
| Diisopropylamide | | | | DMSO, dioxane, heptane, |
| | | | | pentane, TBME, THF, toluene |
| Li/Na/K | 26 (THF) | −12 | 30 | Diethyl ether, diglyme, |
| HMDS | | | | DMSO, dioxane, heptane, |
| | | | | pentane, TBME, THF, toluene |
| Sodium/ | n.d. | n.d. | n.d. | Diglyme, DMSO, dioxane, |
| Potassium tert- | | | | heptane, toluene, triethylamine |
| pentoxide | | | | |
| Sodium/ | 17.0 | −3.0 | 32.2 | Diglyme, DMSO, dioxane, |
| Potassium tert- | | | | heptane, toluene, triethylamine |
| butoxide | | | | |
| Sodium/ | 16.5 | −2.5 | 30.3 | Diglyme, DMSO, dioxane, |
| Potassium | | | | heptane, toluene, triethylamine |
| isopropoxide | | | | |
| Sodium/ | 16.0 | −2.0 | 29.8 | Diglyme, DMSO, dioxane, |
| Potassium | | | | heptane, toluene, triethylamine |
| ethoxide | | | | |
| Lithium/ | 15.7 | −1.7 | 31.4 | Acetonitrile, DMSO, ethanol, |
| Sodium/ | | | | isopropanol, methanol, water |
| Potassium | | | | |
| Hydroxide | | | | |
| Sodium/ | 15.5 | −1.5 | 27.9 | Diglyme, DMSO, dioxane, |
| Potassium | | | | heptane, toluene, triethylamine |
| methoxide | | | | |
| DBU | 12 | 2 | n.d. | Diglyme, DMSO, dioxane, |
| | | | | heptane, toluene |
| N,N- | 11.07 | 2.93 | n.d. | Diglyme, N,N- |
| diisopropylethylamine | | | | diisopropylethylamine, |
| | | | | DMSO, dioxane, heptane, |
| | | | | toluene |
| Diisopropylamine | 11.05 | 2.95 | n.d. | Diglyme, diisopropylamine, |
| | | | | DMSO, dioxane, heptane, |
| | | | | toluene |
| Triethylamine | 10.75 | 3.25 | 9.00 | Diglyme, DMSO, dioxane, |
| | | | | heptane, toluene, triethylamine |
| Sodium/ | 10.3 | 3.7 | n.d. | Diglyme, DMSO, dioxane, |
| Potassium/ | | | | heptane, toluene |
| Cesium | | | | |
| Carbonate | | | | |
| Ammonia | 9.2 | 4.8 | 10.5 | Diglyme, DMSO, dioxane, |
| | | | | heptane, toluene |
| 4- | 9.2 | 4.8 | n.d. | Diglyme, DMSO, dioxane, |
| dimethylamino | | | | |
| pyridine | | | | heptane, toluene |
| (DMAP) | | | | |
| 2,6- | 6.75 | 7.25 | 4.46 | Diglyme, 2,6- |
| dimethylpyridine | | | | dimethylpyridine, DMSO, |
| | | | | dioxane, heptane, toluene |
| Pyridine | 5.21 | 8.79 | 3.4 | Diglyme, DMSO, dioxane, |
| | | | | heptane, pyridine |
| Sodium Amide | 4.7 | 9.3 | 7.9 | Diethyl ether, diglyme, |
| | | | | DMSO, dioxane, heptane, |
| | | | | pentane, TBME, THF, toluene |

Importantly, the term “base” is used in the present disclosure to encompass both reactant-type reactivity and catalyst-type reactivity. In the context of the present disclosure reactant-type reactivity refers to instances where the base is at least partly consumed as reactant is converted to product. In the context of the present disclosure catalyst-type reactivity refers to instances where the base is at least partly consumed as reactant is converted to product the base is not substantially consumed as reactant is converted to product). Also importantly, the term “base” is used in the present disclosure in accordance with Lewis acid/base theory and is not necessarily limited by the base definition(s) provide by Brønsted-Lowery acid/base theory. By way of non-limiting example, the base may comprise sodium tert-butoxide, sodium tert-pentoxide, sodium methoxide, potassium methoxide, sodium ethoxide, potassium ethoxide, sodium isopropoxide, potassium isopropoxide, n-butyllithium, tert-butyllithium, sec-butyllithium, lithium bis(trimethylsilyl)amide, sodium bis(trimethylsilyl)amide, potassium bis(trimethylsilyl)amide, lithium diisopropylamide, lithium diethylamide, sodium hydroxide, potassium hydroxide, calcium hydroxide, sodium hydride, potassium hydride, pyridine, 2,6,-dimethylpyridine, triethylamine, N,N-diisopropylethylamine, diisopropylamine, diethylamine, 1,8-Diazabicyclo[5.4.0]undec-7-ene, sodium amide, 4-dimethylaminopyridine, ammonia, ammonium hydroxide, methylmagnesium bromide, methylmagnesium chloride, sodium carbonate, potassium carbonate, cesium carbonate, or a combination thereof.

In the context of the present disclosure, dimethyl sulfoxide (DMSO) is an organosulfur compound with the formula (CH₃)₂SO. DMSO is typically regarded as a polar aprotic solvent that has the potential to: (i) dissolve both polar and nonpolar compounds; and (ii) form single-phase mixtures with a wide range of organic solvents as well as water.

In the context of the present disclosure, triethylamine (TEA) is an amine compound with the formula N(CH₂CH₃)₃. TEA is typically regarded as a polar aprotic solvent that has the potential to: (i) dissolve both polar and nonpolar compounds; and (ii) form single-phase mixtures with a wide range of organic solvents as well as water (under select temperature conditions).

In select embodiments, the methods of the present disclosure may be conducted in the presence of a co-solvent. The co-solvent may be a class III solvent (such as heptane). By way of non-limiting example, the co-solvent may be acetone, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or combinations thereof. In embodiments that comprise a co-solvent, the ratio of DMSO and/or TEA to co-solvent may range from 1:1 to 1:100.

In select embodiments of the present disclosure, a first cannabinoid is contacted with a base under reaction conditions characterized by: (i) a reaction temperature that is within a target reaction-temperature range for the particular base (the particular solvent system where appropriate), and the first cannabinoid; and (ii) a reaction time that is within a target reaction-time range for the particular base, (the particular solvent system where appropriate), the particular reaction temperature, and the first cannabinoid. As evidenced by the examples of the present disclosure, the basicity of the base (and the characteristics of the solvent system where appropriate) impact the target reaction-temperature range and the target reaction-time range. Importantly, these reaction parameters appear to be dependent variables in that altering one may impact the others. As such, each reaction temperature may be considered in reference to a target reaction-temperature range for the particular base, (the particular solvent system where appropriate), the particular reaction time associated with the reaction, and the first cannabinoid. Likewise, each reaction time in the present disclosure may be considered in reference to a target reaction-time range for the particular base, (the particular solvent system where appropriate) the particular reaction temperature, and the first cannabinoid. With respect to reaction temperatures, by way of non-limiting example, methods of the present disclosure may involve reaction temperatures ranging from about −80° C. to about 200° C. For example, methods of the present disclosure may involve reaction temperatures between: (i) about −80° C. and about 0° C.; (ii) about 0° C. and about 25° C.; (iii) about 25° C. and about 35° C.; (iv) about 35° C. and about 45° C.; (v) about 45° C. and about 55° C.; (vi) about 55° C. and about 65° C.; (vii) about 65° C. and about 75° C.; (viii) about 75° C. and about 85° C.; (ix) about 85° C. and about 95° C.; (x) about 95° C. and about 105° C.; (xi) about 105° C. and about 200° C.; or a combination thereof. Of course, the reaction temperature may be varied over the course of the reaction while still being characterized the one or more of the foregoing reaction temperatures. With respect to reaction times, by way of non-limiting example, methods of the present disclosure may involve reaction temperatures ranging from about 30 minutes to about 85 hours. For example, methods of the present disclosure may involve reaction times between: (i) 30 minutes and about 1 hour; (ii) about 1 hour and about 5 hours; (iii) about 5 hours and about 10 hours; (iv) about 10 hours and 25 hours; (v) about 25 hours and about 40 hours; (vi) about 40 hours and about 55 hours; (vii) about 55 hours and about 70 hours; or (viii) about 70 hours and about 85 hours.

In select embodiments, methods of the present disclosure may involve reactant concentrations ranging from about 0.001 M to about 2 M. For example methods of the present disclosure may involve reactant concentrations of: (i) between about 0.01 M and about 0.1 M; (ii) between about 0.1 M and about 0.5 M; (iii) between about 0.5 M and about 1.0 M; (iv) between about 1.0 M and about 1.5 M; or (v) between about 1.5 M and about 2.0 M.

In select embodiments, methods of the present disclosure may involve base loadings ranges from about 0.1 molar equivalents to about 100 molar equivalents relative to the reactant. For example methods of the present disclosure may involve base loadings of: (i) between about 0.1 molar equivalents to about 1.0 molar equivalents, relative to the reactant; (ii) 0.1.0 molar equivalents to about 5.0 molar equivalents, relative to the reactant; (iii) 5.0 molar equivalents to about 10.0 molar equivalents, relative to the reactant; (iv) 10.0 molar equivalents to about 50.0 molar equivalents, relative to the reactant; or (v) 50.0 molar equivalents to about 100.0 molar equivalents, relative to the reactant.

In select embodiments, the methods of the present disclosure may further comprise a filtering step. By way of non-limiting example the filtering step may employ a fritted Buchner filtering funnel. Suitable filtering apparatus and protocols are within the purview of those skilled in the art.

In select embodiments, the methods of the present disclosure may further comprise a solvent evaporation step, and the solvent evaporation step may be executed under reduced pressure (i.e. in vacuo) for example with a rotary evaporator. Suitable evaporating apparatus and protocols are within the purview of those skilled in the art.

In select embodiments, the polar solvent is an alcohol. Non-limiting examples of alcohols include ethanol, 1-propanol, 2-propanol, butanol, and propylene glycol. In select embodiments, the polar solvent is ethanol, and the basic reagent is sodium ethoxide, potassium ethoxide, or a mixture thereof. In select embodiments, the solvent further comprises sodium hydroxide, potassium hydroxide, or a mixture thereof. A skilled person, having the benefit of the present disclosure, will appreciate that mixtures of ethanol and hydroxide typically form ethoxide anions (including sodium and/or potassium salts thereof) at equilibrium.

### EXAMPLES

### Example 1

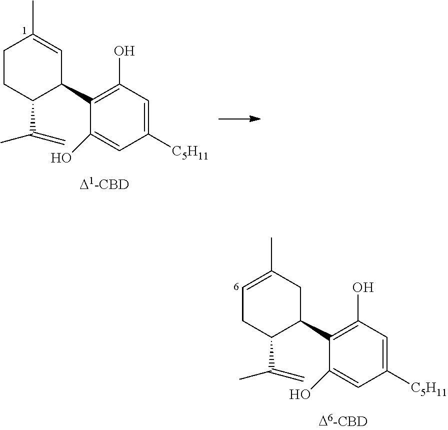

To a solution of Δ¹-CBD (1 g, 3.18 mmol) in reaction solvent (50 mL, DMSO/heptane, 1:5) stirring at room temperature was added potassium tert-butoxide (1.43 g, 12.72 mmol) in a portion-wise manner. The reaction was heated to reflux and stirred for 2 hours. Reaction progress was monitored by TLC. Following reaction completion, the reaction vessel was cooled to room temperature and was transferred to an ice bath. 1N HCl (aq) was added dropwise with stirring until the excess base was quenched. The reaction mixture was transferred to a separatory funnel and was diluted with 1:1 water/tert-butyl methyl ether (TBME). The layers were partitioned and the aqueous layer was extracted twice more with TBME. The organic layers were combined, washed with saturated sodium chloride, dried with sodium sulfite, and volatiles were concentrated in vacuo. Analysis by HPLC (DAD 215 nm) indicated the presence of a compound eluted as expected for Δ⁶-CBD.

### Example 2

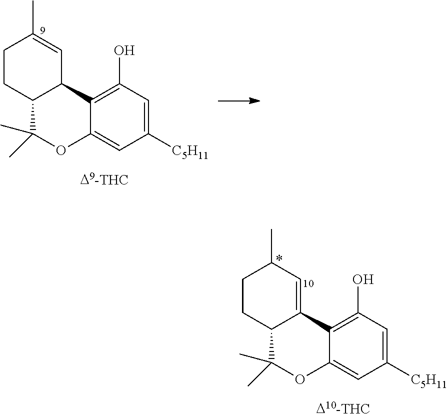

To a solution of Δ⁹-THC (1 g, 3.18 mmol) in reaction solvent (50 mL, DMSO/heptane, 1:5) stirring at room temperature was added potassium tert-butoxide (1.43 g, 12.72 mmol) in a portion-wise manner. The reaction was heated to reflux and stirred for 2 hours. Reaction progress was monitored by TLC. Following reaction completion, the reaction vessel was cooled to room temperature and was transferred to an ice bath. 1N HCl (aq) was added dropwise with stirring until the excess base was quenched. The reaction mixture was transferred to a separatory funnel and was diluted with 1:1 water/tert-butyl methyl ether (TBME). The layers were partitioned and the aqueous layer was extracted twice more with TBME. The organic layers were combined, washed with saturated sodium chloride, dried with sodium sulfite, and volatiles were concentrated in vacuo. Analysis by HPLC (DAD 215 nm) indicated the presence of a compound eluted as expected for Δ¹⁰-THC.

### Example 3

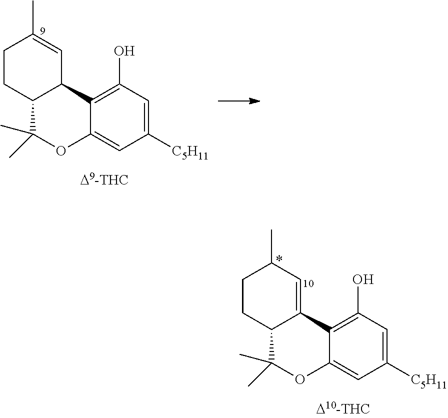

To a flask containing Δ⁹-THC (4.88 g, 15.6 mmol, 1.0 equiv., ˜90% purity) under N₂ was added solid potassium tert-butoxide (12.0 g, 109 mmol, 7 equiv.), DMSO (20 mL) and toluene (50 mL). The mixture was stirred and heated to 110° C. for 2 h under N₂. The flask was cooled to room temperature, and quenched with 10% wt/wt aq. citric acid with vigorous stirring for 10-30 min. The layers were separated and the aqueous layer was extracted with methyl t-butyl ether (MTBE). The combined organic layers were washed with water, dried over Na₂SO₄, and evaporated to give a dark red oil that crystallized on standing. Alternatively, the residue was purified by flash column chromatography on silica. Elution with MTBE in heptane provided trans-Δ¹⁰-THC as a yellow oil that crystallized (3.7 g, 76% yield). Further chromatographic elution yielded cis-Δ¹⁰-THC as colourless crystals (0.377 g, 8% yield).

### Example 4

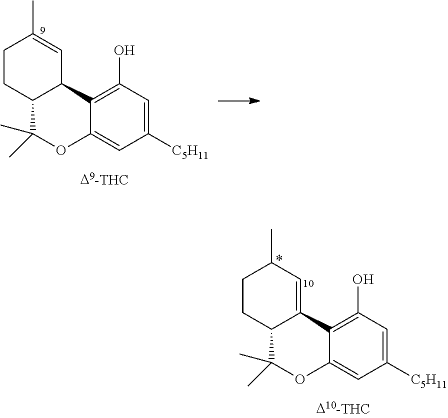

Δ⁹-THC was converted to Δ¹⁰-THC in accordance with a method of the present disclosure and the conditions outlined in Table 2. In particular, the polar solvent and/or the co-solvent was varied.

Generally, the reactions were performed out as follows: To a tube containing Δ⁹-THC (0.25-0.4 g, 78% purity, ˜1 mmol, 1.0 equiv.) under N₂ was added solid potassium tert-butoxide. Co-solvent (about 10 mass equiv.) and polar solvent (about 5 mass equiv.) were added and the mixture was heated with stirring for a given time. The mixture was cooled to room temperature and quenched with excess 10% wt/wt aq. citric acid, with vigorous stirring for 10-30 min under N₂. The mixture was diluted with heptane and/or MTBE, the layers were separated, and the organic layer washed twice with water. Evaporation of solvents under vacuum provided a mixture that was analyzed by HPLC

The amount of Δ⁹-THC remaining after the reaction is complete, and the composition of the purified product for each reaction, is reported in Table 2.

TABLE 2
Summary results for Example 4

| | | | | equiv. | | Δ9 THC | cis-Δ10 | trans-Δ10 | |
|---|---|---|---|---|---|---|---|---|---|
| | Temp. | Co- | Polar | KOtBu | Time | remaining | THC | THC | selectivity |
| Entry | (° C.) | solvent | Solvent | to THC | (h) | (w/w %) | w/w % | w/w % | (trans:cis) |
| 1 | 130 | Heptane | — | 2.5 | 1 | 71.7 | 9.8 | 36.6 | 3.7 |
| 2 | 110 | Toluene | DMSO | 8.5 | 2 | 0.5 | 5.4 | 28.1 | 5.2 |
| 3 | 80 | Toluene | DMSO | 7 | 19 | 4.5 | 6.9 | 44.6 | 6.5 |
| 4 | 100 | Anisole | DMSO | 7 | 1.5 | 10.3 | 6.8 | 43.2 | 6.4 |
| 5 | 100 | Heptane | TEA | 7 | 1.5 | 17.5 | 6.9 | 36.8 | 5.3 |

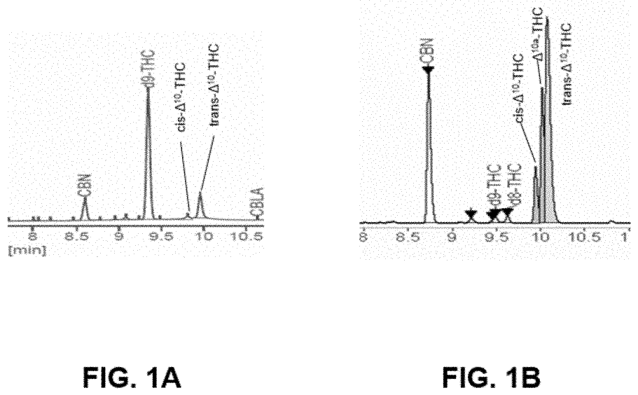

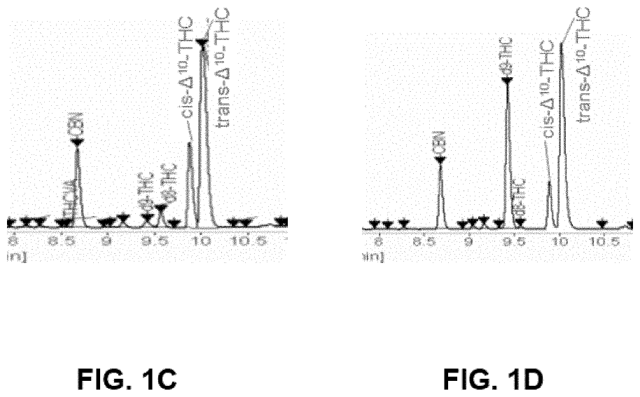

HPLC chromatograms of the output material from entries 1, 2, 4, and 5 are set out in FIG. 1A, FIG. 1B, FIG. 1C, and FIG. 1D, respectively. FIG. 1A, indicates that the presence of polar solvent is necessary to achieve significant isomerization of Δ⁹-THC. FIGS. 1B, 1C, and 1D indicate that heptane and anisole are suitable co-solvents and that DMSO, and TEA are effective co-solvents for preparing Δ¹⁰-THC by base-promoted double bond migration. Notably, heptane, anisole, DMSO, and TEA are class III solvents.

### Example 5

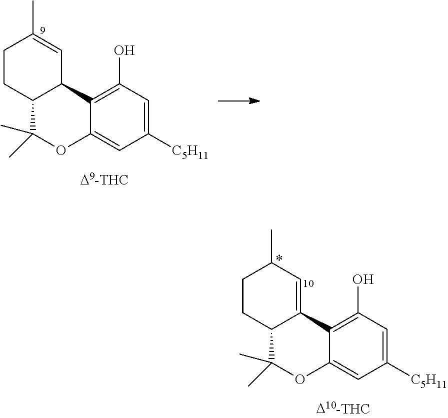

Δ⁹-THC was converted to Δ¹⁰-THC in accordance with a method of the present disclosure and the conditions outlined in Table 3. In particular, the polar solvent and/or the co-solvent was varied.

Generally, the reactions were performed as follows: To a flask containing a Δ⁹-THC (0.25-0.4 g, 78% purity, ˜1 mmol, 1.0 equiv.) under N₂ was added a solid base. Solvent and co-solvent (about 5-15 mass equiv. total) were added, and the mixture was heated with stirring for a given time. The mixture was cooled to room temperature and quenched with excess acetic acid or 10% wt/wt aq. citric acid, with vigorous stirring for 10-30 min under N₂. The mixture was diluted with heptane and/or MTBE, the layers were separated, and the organic layer washed twice with water. Evaporation of solvents under vacuum provided a mixture that was analyzed by HPLC.

In the case of lithium diisopropylamide (LDA), the general procedure was as follows: To a solution of LDA (1 M in THF; 4.4 mL, 4.4 mmol) under N₂ was added dropwise a solution of Δ⁹-THC (277 mg, 78% purity, 0.68 mmol) in solvent (MTBE or NEt₃, 5 mL), and the mixture heated to 45° C. for 72 h. The mixture was quenched with excess aq. citric acid, diluted with heptane, and the layers were separated. The organic layer was washed twice with water, concentrated in vacuo, and analyzed by HPLC.

The amount of Δ⁹-THC remaining after the reaction is complete, and the composition of the purified product for each reaction, is reported in Table 3.

TABLE 3
Summary results for Example 5.

| | | | | | | | Δ9 THC | cis-Δ10 | trans-Δ10 | |
|---|---|---|---|---|---|---|---|---|---|---|
| | Temp. | Co- | Polar | Basic | equiv. | Time | remaining | THC | THC | selectivity |
| Entry | (° C.) | solvent | Solvent | reagent | to THC | (h) | (w/w %) | w/w % | w/w % | (trans:cis) |
| 1 | 80 | — | EtOH | KOH | 45 | 19 | 62.8 | 1.5 | 18.3 | 12.6 |
| 2 | 110 | Toluene | — | NaH | 15 | 2 | 42.3 | 0 | 0 | n/a |
| 3 | 100 | Heptane | — | MgO | 0.7 by | 20 | 69.7 | 0 | trace | n/a |
| | | | | | mass | | | | | |
| 4 | 80 | Toluene | DMSO | KOtBu | 7 | 19 | 4.5 | 6.9 | 44.6 | 6.5 |
| 5 | 125 | — | DMSO | KOH | 7 | 1.5 | 84.1 | 0.4 | 1.6 | 4.4 |
| 6 | 45 | TBME | — | LDA | 5 | 72 | 77.1 | 0 | trace | n/a |
| 7 | 45 | — | NEt₃ | LDA | 5 | 72 | 71.0 | 0 | trace | n/a |

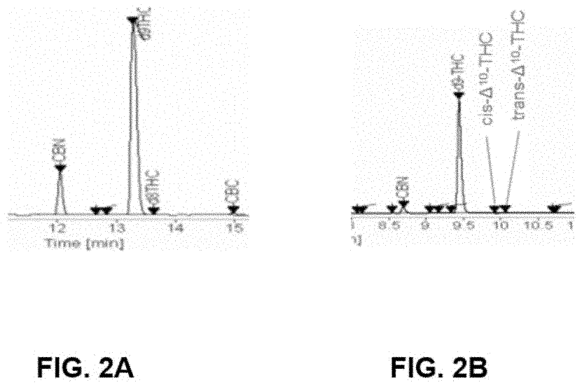

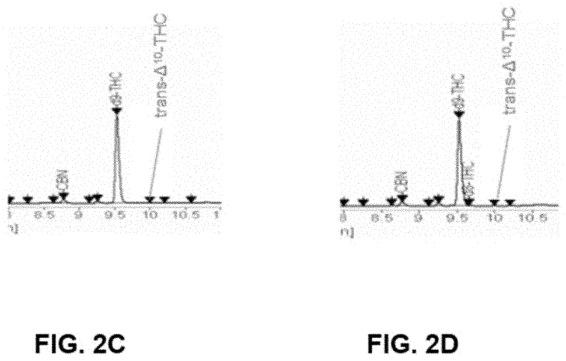

HPLC chromatograms of the output material from entries 2, 5, 6, and 7 are set out in FIG. 2A, FIG. 2B, FIG. 2C, and FIG. 2D, respectively. FIGS. 2A to 2D indicate that sodium hydride (NaH), lithium diisopropylamide, and KOH in the absence of ethanol are not sufficiently basic to promote double-bond migration. Notably, KOH in ethanol is sufficiently basic to promote double-bond migration, which may be due to the formation of ethoxide anions by deprotonation of ethanol by KOH.

### Example 6

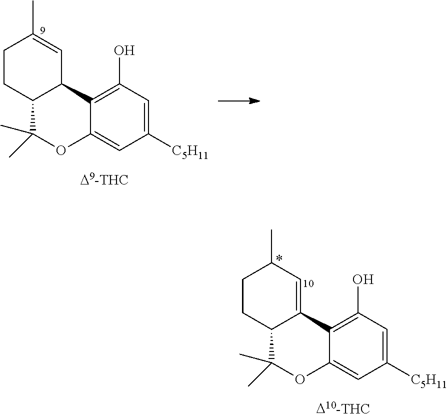

Δ⁹-THC was converted to Δ¹⁰-THC in accordance with a method of the present disclosure and the conditions outlined in Table 4 and the general procedures of Example 5. In particular, the reaction time, reaction temperature, and the co-solvent was varied. The amount of Δ⁹-THC remaining after the reaction is complete, and the composition of the purified product for each reaction, is reported in Table 4.

TABLE 4
Summary results for Example 6.

| | | | | | | | Δ9 THC | cis-Δ10 | trans-Δ10 | |
|---|---|---|---|---|---|---|---|---|---|---|
| | Temp. | Co- | Polar | Basic | equiv. | Time | remaining | THC | THC | selectivity |
| Entry | (° C.) | solvent | Solvent | reagent | to THC | (h) | (w/w %) | w/w % | w/w % | (trans:cis) |
| 1 | 110 | Toluene | DMSO | KOtBu | 8.5 | 2 | 0.5% | 5.4 | 28.1 | 5.2 |
| 2 | 80 | Toluene | DMSO | KOtBu | 7 | 1.25 | 82.1% | 0.87 | 9.4 | 10.8 |
| 3 | 80 | Toluene | DMSO | KOtBu | 7 | 19 | 4.5% | 6.9 | 44.7 | 6.5 |
| 4 | 80 | Toluene | DMSO | KOtBu | 7 | 25 | 2.6% | 5.9 | 42.0 | 7.0 |
| 5 | 100 | Anisole | DMSO | KOtBu | 7 | 1.5 | 10.3% | 6.8 | 43.2 | 6.4 |
| 6 | 125 | Anisole | DMSO | KOtBu | 7 | 1.5 | 0.5% | 11.6 | 44.6 | 3.8 |

As shown in Table 4, the selectivity of the reaction with regard to the ratio of trans-Δ¹⁰-THC:cis-Δ¹⁰-THC in the product is affected by the reaction time, the reaction temperature, and the co-solvent.

### Example 7

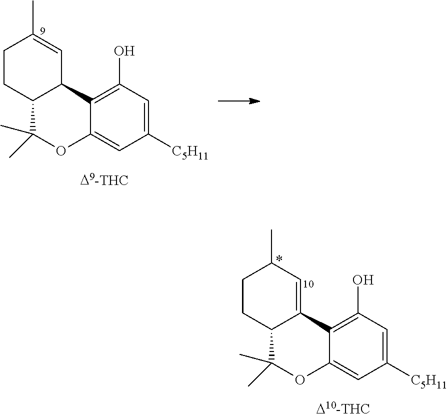

To a flask containing potassium tert-butoxide (11.9 g, 106 mmol, 6.6 equiv.) and triethylamine (32 mL, 224 mmol, 14 equiv.) under N₂ was added a solution of □⁹-THC resin (7.0 g, 73% purity, 16 mmol, 1.0 equiv.) in heptane (20 mL), and the mixture was refluxed at 105° C. under N₂ for 1.5 h. Another 30 mL portion of heptane was added and the mixture was heated an additional 1 h. The mixture was cooled, water was added slowly under N₂, and the mixture was quenched with 50% wt/wt aq. citric acid, with vigorous stirring for 10-30 min under N₂. The mixture was diluted with heptane, and the layers were separated. The organic layer was washed with water, dried over Na₂SO₄, and evaporated to give a dark brown oil (8.0 g) that crystallized after prolonged standing at −80° C. Filtration of the crystals and recrystallization gave cis-Δ¹⁰-THC as white needles (227 mg, 5% yield). The remainder of the resin was separated by dry column vacuum chromatography on silica, eluted with MTBE in heptane to give trans-Δ¹⁰-THC as a brown oil that crystallized on prolonged standing. Analysis by HPLC (DAD 215 nm) indicated the presence of a compound eluted as expected for Δ¹⁰-THC.

### Example 8

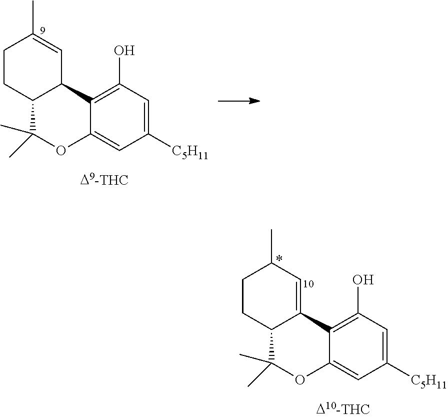

To a tube containing Δ⁹-THC (0.412 g, 78% purity, 1.02 mmol, 1.0 equiv.) under N₂ was added solid potassium tert-butoxide (0.99 g, 8.9 mmol, 8.7 equiv.), and the tube flushed with N₂. Anisole (4.5 mL) and DMSO (2.3 mL) were added, and the mixture was stirred and heated to 125° C. for 1.5 h. The mixture was cooled to room temperature and quenched with 10% wt/wt aq. citric acid, with vigorous stirring for 10-30 min under N₂. The mixture was diluted with heptane, the layers were separated, and the organic layer washed. Evaporation of heptane gave 2.374 g solution containing mainly cis-Δ¹⁰-THC and trans-Δ¹⁰-THC in a 1:3.8 ratio as shown by HPLC analysis.

### Example 9

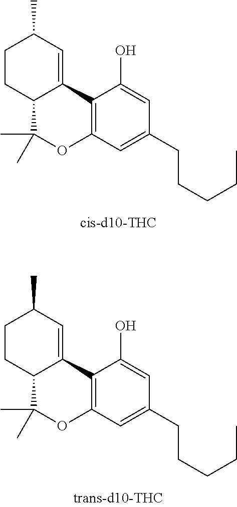

Single crystals of cis-Δ¹⁰-THC and of trans-Δ¹⁰-THC were each grown by slow cooling of heptane solution.

A single crystal of trans-Δ¹⁰-THC was mounted on a Mitegen polyimide micromount with a small amount of Paratone N oil for X-ray crystallography analysis. All X-ray measurements were made on a Bruker Kappa Axis Apex2 diffractometer at a temperature of 110 K. The unit cell dimensions were determined from a symmetry constrained fit of 9984 reflections with 6.16°<2θ<54.74°. The data collection strategy was a number of ω and φ scans which collected data up to 54.956° (2θ). The frame integration was performed using SAINT. The resulting raw data was scaled and absorption corrected using a multi-scan averaging of symmetry equivalent data using SADABS.

The structure was solved by using a dual space methodology using the SHELXT program. All non-hydrogen atoms were obtained from the initial solution. The hydrogen atoms were introduced at idealized positions. The oxygen bound hydrogen was allowed to refine isotropically while all the carbon bound hydrogen atoms were constrained to ride on their respective parent atoms. The structural model was fit to the data using full matrix least-squares based on F2. The calculated structure factors included corrections for anomalous dispersion from the usual tabulation. The structure was refined using the SHELXL program from the SHELX suite of crystallographic software. Graphic plots were produced using the Mercury program.

A single crystal of cis-Δ¹⁰-THC was mounted on a Mitegen polyimide micromount with a small amount of Paratone N oil. All X-ray measurements were made on a Bruker Kappa Axis Apex2 diffractometer at a temperature of 110 K. The unit cell dimensions were determined from a symmetry constrained fit of 5821 reflections with 5.22° <2θ<52.78°. The data collection strategy was a number of ω and φ scans which collected data up to 57.208° (2θ). The frame integration was performed using SAINT. The resulting raw data was scaled and absorption corrected using a multi-scan averaging of symmetry equivalent data using SADABS.

The structure was solved by using a dual space methodology using the SHELXT program. All non-hydrogen atoms were obtained from the initial solution. The hydrogen atoms were introduced at idealized positions and were allowed to refine isotropically. The absolute configuration was assigned in consultation with the sample originator. The structural model was fit to the data using full matrix least-squares based on F2. The calculated structure factors included corrections for anomalous dispersion from the usual tabulation. The structure was refined using the SHELXL program from the SHELX suite of crystallographic software. Graphic plots were produced using the Mercury program.

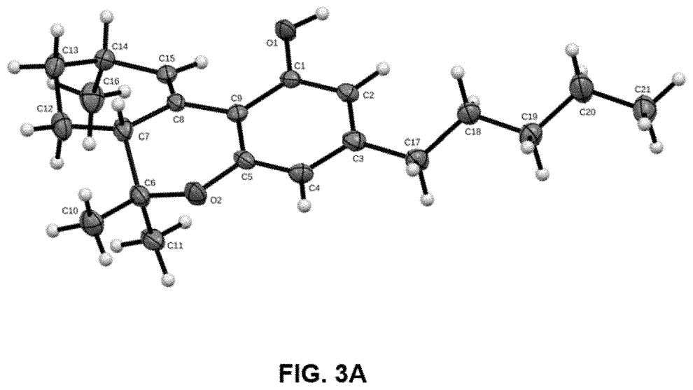

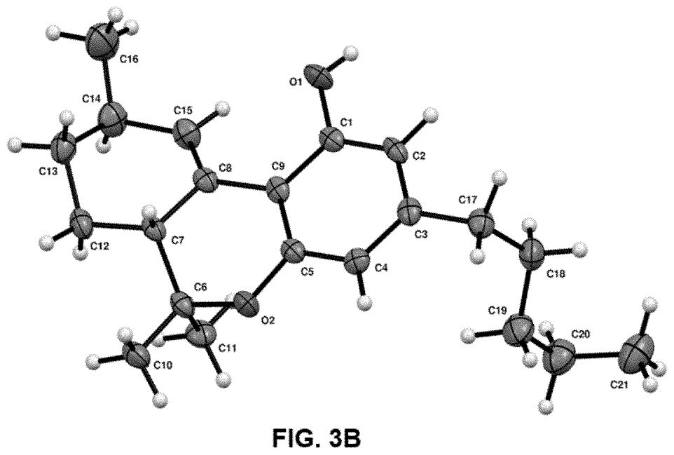

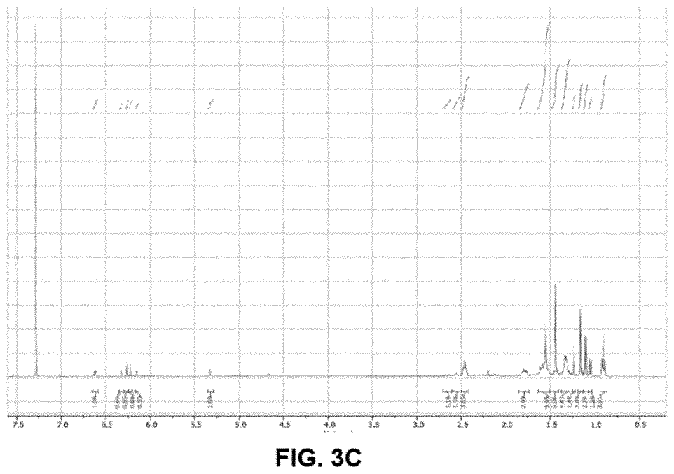

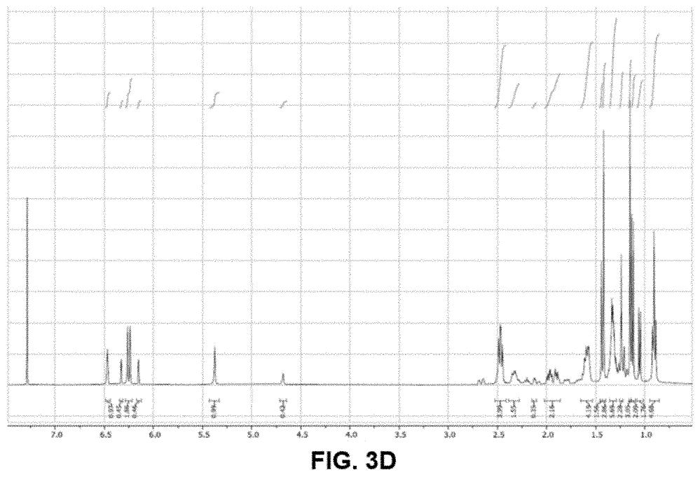

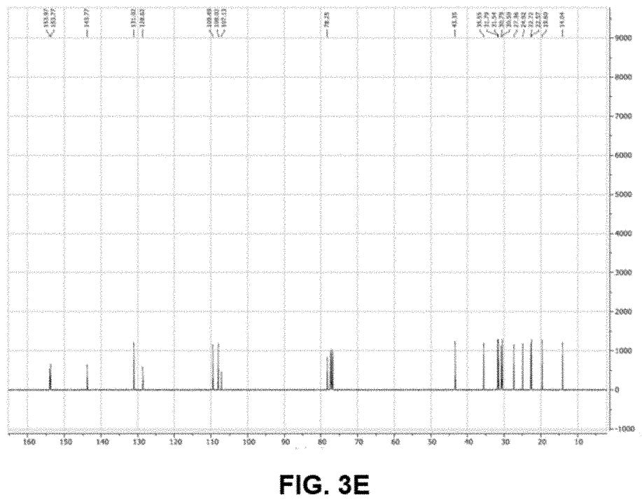

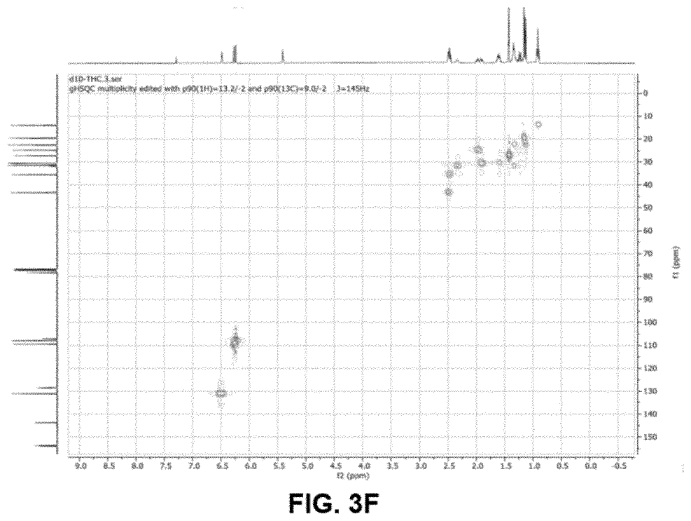

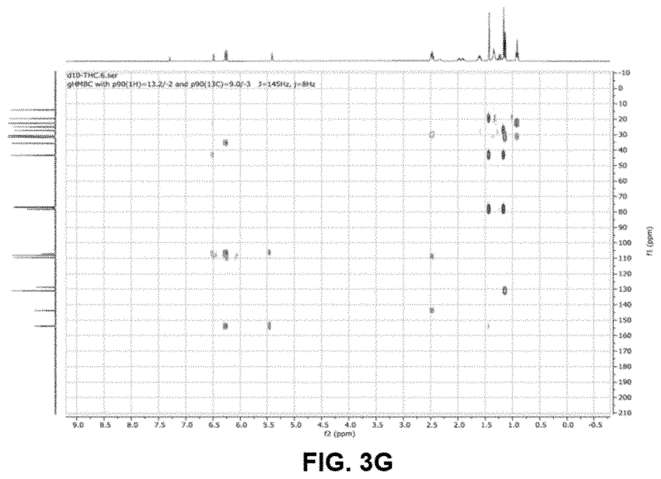

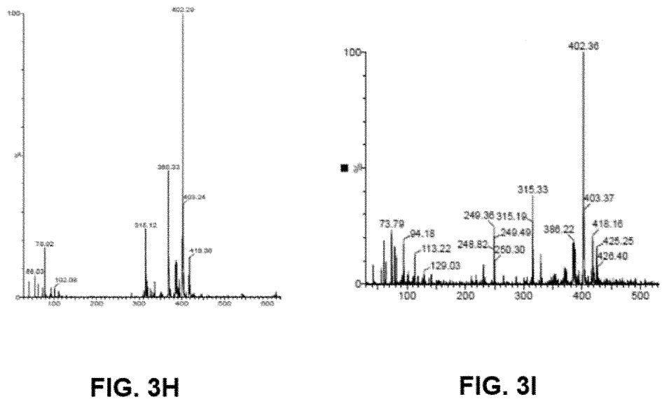

A representative graphic plot of the crystal structures of cis-Δ¹⁰-THC and trans-Δ¹⁰-THC are shown in FIGS. 3A and 3B, respectively. ¹H NMR spectra of cis-Δ¹⁰-THC and trans-Δ¹⁰-THC are shown in FIG. 3C and FIG. 3D, respectively. ¹³C, heteronuclear single quantum coherence spectroscopy (HSQC), and heteronuclear multiple bond correlation (HMBC) NMR spectra for trans-Δ¹⁰-THC are showing in FIGS. 3E, 3F and 3G, respectively. Mass spectra of cis-Δ¹⁰-THC and trans-Δ¹⁰-THC are shown in FIG. 3H and FIG. 3I, respectively.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1.-18. (canceled)

19. A method for converting a first cannabinoid into a second cannabinoid that is a regioisomer of the first cannabinoid, the method comprising contacting the first cannabinoid with a base in a solvent system comprising a polar solvent.

20. The method of claim 19, wherein the polar solvent is dimethyl sulfoxide (DMSO), triethylamine (TEA), or a combination thereof.

21. The method of claim 19, wherein the polar solvent is ethanol.

22. The method of claim 19, wherein the base is sodium tert-butoxide, sodium tert-pentoxide, sodium methoxide, potassium methoxide, sodium ethoxide, potassium ethoxide, sodium isopropoxide, potassium isopropoxide, n-butyllithium, tert-butyllithium, sec-butyllithium, lithium bis(trimethylsilyl)amide, sodium bis(trimethylsilyl)amide, potassium bis(trimethylsilyl)amide, lithium diisopropylamide, lithium diethylamide, sodium hydroxide, potassium hydroxide, calcium hydroxide, sodium hydride, potassium hydride, pyridine, 2,6,-dimethylpyridine, triethylamine, N,N-diisopropylethylamine, diisopropylamine, diethylamine, 1,8-Diazabicyclo[5.4.0]undec-7-ene, sodium amide, 4-dimethylaminopyridine, ammonia, ammonium hydroxide, methylmagnesium bromide, methylmagnesium chloride, sodium carbonate, potassium carbonate, cesium carbonate, or any combination thereof.

23. The method of claim 19, wherein the base is potassium tert-butoxide.

24. The method of claim 19, wherein the solvent system further comprises a co-solvent.

25. The method of claim 24, wherein the co-solvent is a class III solvent.

26. The method of claim 24, wherein co-solvent is heptane, tert-butyl methyl ether, anisole, cumene, toluene, tetrahydrofuran, dioxane, dimethyl formamide, dimethyl acetamide, N-methyl pyrrolidone, hexane, octane, acetonitrile, cyclohexane, ortho-xylene, meta-xylene, para-xylene, or any combination thereof.

27. The method of claim 24, wherein the solvent system comprises the polar solvent and the co-solvent at a polar solvent:co-solvent ratio of between about 1:1 and about 1:20.

28. The method of claim 19, wherein the first cannabinoid has a concentration of between about 0.1 M and about 1 M with respect to the solvent system.

29. The method of claim 19, wherein the base has a reagent loading of between about 1 molar equivalent and about 50 molar equivalents with respect to the first cannabinoid.

30. A method for converting Δ¹-cannabidiol (Δ¹-CBD) into Δ⁶-cannabidiol (Δ⁶-CBD), the method comprising contacting the Δ¹-CBD with a base in a solvent system comprising a polar solvent.

31. The method of claim 30, wherein the solvent system further comprises a co-solvent.

32. The method of claim 31, wherein the solvent system comprises dimethyl sulfoxide (DMSO) and heptane.

33. The method of claim 30, wherein the base is sodium tert-butoxide, sodium tert-pentoxide, sodium methoxide, potassium methoxide, sodium ethoxide, potassium ethoxide, sodium isopropoxide, potassium isopropoxide, n-butyllithium, tert-butyllithium, sec-butyllithium, lithium bis(trimethylsilyl)amide, sodium bis(trimethylsilyl)amide, potassium bis(trimethylsilyl)amide, lithium diisopropylamide, lithium diethylamide, sodium hydroxide, potassium hydroxide, calcium hydroxide, sodium hydride, potassium hydride, pyridine, 2,6,-dimethylpyridine, triethylamine, N,N-diisopropylethylamine, diisopropylamine, diethylamine, 1,8-Diazabicyclo[5.4.0]undec-7-ene, sodium amide, 4-dimethylaminopyridine, ammonia, ammonium hydroxide, methylmagnesium bromide, methylmagnesium chloride, sodium carbonate, potassium carbonate, cesium carbonate, or any combination thereof.

34. The method of claim 30, wherein the base is potassium tert-butoxide.

35. A method for converting Δ⁹-tetrahydrocannabinol (Δ⁹-THC) into Δ¹⁰-tetrahydrocannabinol (Δ¹⁰-THC), the method comprising contacting the Δ⁹-THC with a base in a solvent system comprising a polar solvent.

36. The method of claim 35, wherein the polar solvent is dimethyl sulfoxide (DMSO), triethylamine (TEA), or a combination thereof.

37. The method of claim 35, wherein the polar solvent is ethanol.

38. The method of claim 35, wherein the solvent system further comprises a co-solvent.

39. The method of claim 38, wherein co-solvent is heptane, tert-butyl methyl ether, anisole, cumene, toluene, tetrahydrofuran, dioxane, dimethyl formamide, dimethyl acetamide, N-methyl pyrrolidone, hexane, octane, acetonitrile, cyclohexane, ortho-xylene, meta-xylene, para-xylene, or any combination thereof.

40. The method of claim 35, wherein the base is sodium tert-butoxide, sodium tert-pentoxide, sodium methoxide, potassium methoxide, sodium ethoxide, potassium ethoxide, sodium isopropoxide, potassium isopropoxide, n-butyllithium, tert-butyllithium, sec-butyllithium, lithium bis(trimethylsilyl)amide, sodium bis(trimethylsilyl)amide, potassium bis(trimethylsilyl)amide, lithium diisopropylamide, lithium diethylamide, sodium hydroxide, potassium hydroxide, calcium hydroxide, sodium hydride, potassium hydride, pyridine, 2,6,-dimethylpyridine, triethylamine, N,N-diisopropylethylamine, diisopropylamine, diethylamine, 1,8-Diazabicyclo[5.4.0]undec-7-ene, sodium amide, 4-dimethylaminopyridine, ammonia, ammonium hydroxide, methylmagnesium bromide, methylmagnesium chloride, sodium carbonate, potassium carbonate, cesium carbonate, or any combination thereof.

41. The method of claim 35, wherein the base is potassium tert-butoxide.
